(19) United States
(12) Patent Application Publication
(10) Pub. No.: US 2026/0272726 A1
(43) Pub. Date: Sep. 17, 2026

(54) ABSORBENT ARTICLE

(71) Applicant: KNH ENTERPRISE CO., LTD., Tainan City (TW)

(72) Inventors: Cheng-Tai HUANG, Tainan City (TW); . SHU, Tainan City (TW); Xiangyu YAN, Tainan City (TW)

(21) Appl. No.: 19/294,750

(22) Filed: Aug. 8, 2025

(30) Foreign Application Priority Data
Mar. 13, 2025 (CN) 202510297398.8

(51) Int. Cl. A61F 13/475; A61F 13/514

(52) U.S. Cl. CPC A61F 13/4757; A61F 13/51401; A61F 2013/51411

(57) ABSTRACT

An absorbent article includes a liquid-impermeable underlayer, an absorbent unit and two folded units. The liquid-impermeable underlayer is for preventing penetration of a liquid. The absorbent unit is disposed on the liquid-impermeable underlayer in a first direction for absorbing the liquid, and includes a main portion and two sides portions which are respectively connected to two opposite sides of the main portion in a second direction. Each of the folded units includes a side barrier structure and a supporting structure. The side barrier structures of the folded units are respectively located on outer sides of the side portions and are adapted to prevent the liquid from flowing outwardly. The supporting structure of each of the folded units is disposed between the liquid-impermeable underlayer and an adjacent one of the side portions so that the main portion is spaced apart from the liquid-impermeable underlayer by a gap.

### CROSS-REFERENCE TO RELATED APPLICATION

This application claims priority to Chinese Invention Patent Application No. 202510297398.8, filed on March 13, 2025, the entire disclosure of which is incorporated by reference herein.

### FIELD

The disclosure relates to an absorbent article for human life necessity, and more particularly to an absorbent article with folded units for external application.

### BACKGROUND

An absorbent article, such as a sanitary napkin or a diaper, includes a waterproof underlayer, an absorbent body which is layered on the waterproof underlayer, and a leakproof structure which covers the waterproof underlayer and side portions of the absorbent body. During use, the absorbent article is placed in contact with a user’s skin, the absorbent body is used to absorb body fluids, and the waterproof underlayer and the leakproof structure are used to prevent body fluids from leaking out from the absorbent body. However, after the absorbent body absorbs the body fluids, some of the body fluids naturally vaporizes into vapor but is blocked from escaping outwardly by the waterproof underlayer which is attached to the absorbent body, leading to a feeling of stuffiness when using the conventional absorbent article. Moreover, when the conventional absorbent article is used as a sanitary napkin, the waterproof underlayer thereof is usually adhered to the user’s clothing so as to fix its position. However, the absorbent body of the conventional absorbent article which is layered on the waterproof underlayer may shift due to the user’s movements, causing displacement of the conventional absorbent article during use, which may result in leakage of the body fluids.

### SUMMARY

Therefore, an object of the disclosure is to provide an absorbent article that can alleviate at least one of the drawbacks of the prior art.

According to the disclosure, the absorbent article includes a liquid-impermeable underlayer, an absorbent unit and two folded units. The liquid-impermeable underlayer is for preventing penetration of a liquid. The absorbent unit is disposed on the liquid-impermeable underlayer in a first direction for absorbing the liquid, and includes a main portion and two side portions which are respectively connected to two opposite sides of the main portion in a second direction that intersects the first direction. Each of the folded units includes a side barrier structure and a supporting structure which is connected to the side barrier structure. The side barrier structures of the folded units are respectively located on outer sides of the side portions which are distal from each other and are adapted to prevent the liquid from flowing outwardly from the absorbent unit. The supporting structure of each of the folded units is disposed between the liquid-impermeable underlayer and an adjacent one of the side portions so that the main portion is spaced apart from the liquid-impermeable underlayer by a gap.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a perspective view illustrating an embodiment of an absorbent article with folded units according to the disclosure.

FIG. 2 is a cross-sectional view taken along line II-II of FIG. 1.

FIG. 3 is a partially enlarged cross-sectional view of the embodiment.

FIG. 4 is a partially enlarged cross-sectional view similar to FIG. 3, illustrating another configuration of supporting structures of the embodiment.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

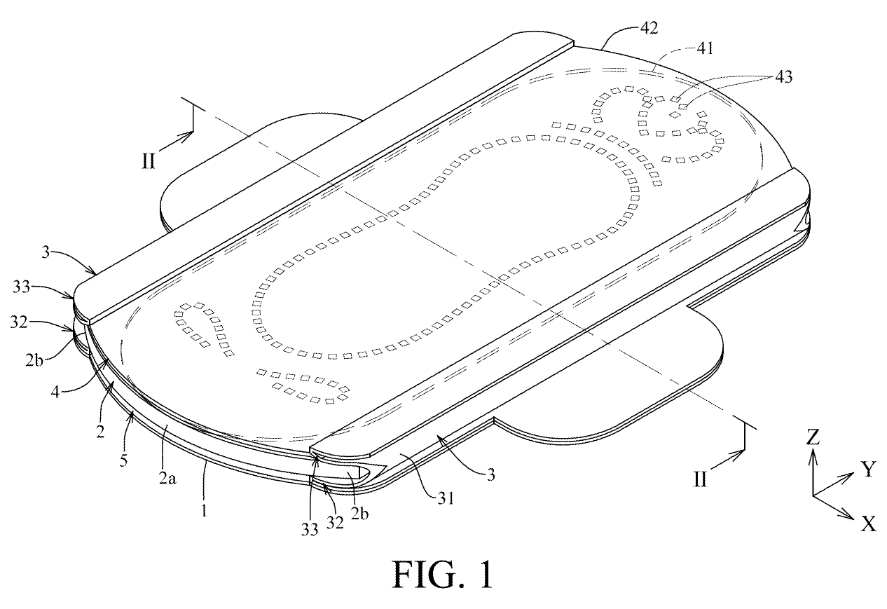

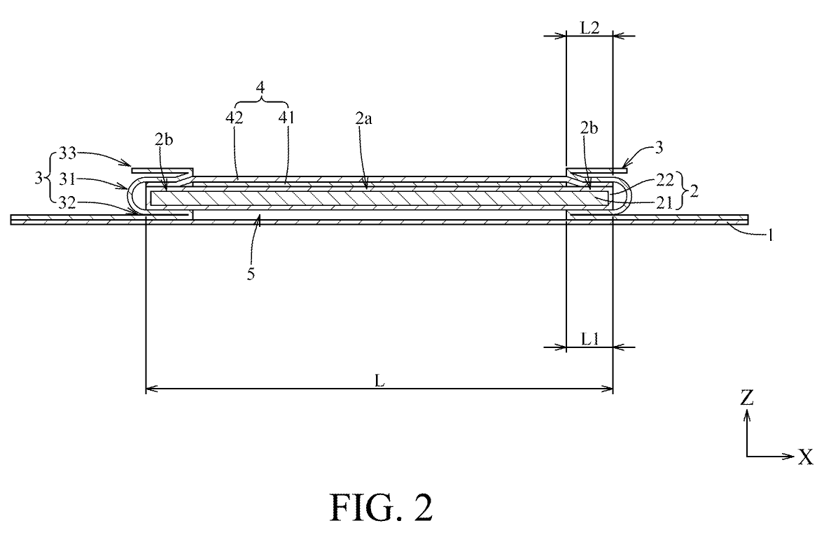

FIGS. 1 and 2 illustrate an embodiment of an absorbent article according to the disclosure. For convenience of description, a first direction (Z), a second direction (X) which is not parallel to the first direction (Z), and a third direction (Y) which is not parallel to the first direction (Z) and the second direction (X) are defined based on use environment of the absorbent article. In the embodiment, the first direction (Z), the second direction (X) and the third direction (Y) are perpendicular to one another. The first direction (Z) is a top-bottom direction as indicated by an arrow (Z) in FIG. 1. The second direction (X) is a left-right direction as indicated by an arrow (X) in FIG. 1. The third direction (Y) is a front-rear direction as indicated by an arrow (Y) in FIG. 1.

The absorbent article may be a sanitary napkin or a diaper, but is not limited thereto, and is used to absorb body fluids such as menstrual blood and urine (which are referred to as liquid hereinafter). In the embodiment, the absorbent article is exemplified by the sanitary napkin. The absorbent article includes a liquid-impermeable underlayer 1, an absorbent unit 2, two folded units 3 and a top sheet structure 4. The liquid-impermeable underlayer 1 is a strip-like membrane which extends in the second direction (X) and the third direction (Y). An upper surface of the liquid-impermeable underlayer 1 is closer to a user’s skin than a lower surface of the liquid-impermeable underlayer 1. The liquid-impermeable underlayer 1 is used to prevent liquid from penetration and flowing downwardly. In some embodiments, the liquid-impermeable underlayer 1 has a plurality of holes which are gas-permeable but liquid-impermeable (not shown in the figure) such that the absorbent article is gas-permeable, thereby preventing the feeling of stuffiness and enhancing a user’s comfort during use. However, the liquid-impermeable underlayer 1 may also be a gas-impermeable film, depending on actual needs.

The absorbent unit 2 is disposed on the liquid-impermeable underlayer 1 in the first direction (Z) for absorbing the liquid, and has a main portion (2a) and two side portions (2b). In this embodiment, the absorbent unit 2 includes an absorbent body 21 and a wrapping layer 22 which wraps the absorbent body 21. The absorbent body 21 and the wrapping layer 22 cooperatively define the main portion (2a) and the side portions (2b) which are respectively connected to two opposite sides of the main portion (2a) in the second direction (X) that intersects the first direction (Z). In other words, the side portions (2b) respectively extend away from each other from the two opposite sides of the main portion (2a) in the second direction (X). The main portion (2a) and the side portions (2b) which are defined by the absorbent body 21 are integrally formed in one piece. The main portion (2a) and the side portions (2b) which are defined by the wrapping layer 22 are integrally formed in one piece. In the embodiment, a material of the absorbent body 21 includes a mixture of superabsorbent polymer (SAP) and pulp. For instance, the material of the absorbent body 21 may be a composite of the pulp and granular SAP distributed in the pulp. A material of the wrapping layer 22 includes nonwoven fabric or plant fiber (e.g., pulp). However, the materials of the absorbent body 21 and the wrapping layer 22 are not limited to the above descriptions.

The top sheet structure 4 covers the absorbent unit 2 and is liquid-permeable for guiding the liquid towards the absorbent unit 2. The top sheet structure 4 includes a fluid distribution layer 41 which is disposed on the absorbent unit 2, and a skin contact layer 42 which covers the fluid distribution layer 41 and the side portions (2b). A dimension (width) of the skin contact layer 42 in the second direction (X) is greater than a dimension (width) of the fluid distribution layer 41 in the second direction (X). The skin contact layer 42 is to be in contact with the user’s skin, is liquid-permeable and allows the liquid to penetrate therethrough to the fluid distribution layer 41. The fluid distribution layer 41 is used to guide the liquid from the skin contact layer 42 towards the absorbent unit 2. The top sheet structure 4 prevents the user’s skin from directly contacting the absorbent unit 2 so as to keep the user’s skin dry. Furthermore, as shown in FIG. 1, the top sheet structure 4 is formed with recessed portions 43 which are spaced apart from each other and which are recessed from an upper surface of the top sheet structure 4 to the absorbent unit 2. The recessed portions 43 may be textures or patterns which are formed by heat pressing the top sheet structure 4, or may be through holes. The recessed portions 43 allow the liquid to permeate into the absorbent unit 2 rapidly. In the embodiment, a material of the skin contact layer 42 includes spunbond nonwoven fabric, thermal bonded nonwoven fabric, a perforated film or cotton fabric. A material of the fluid distribution layer 41 includes thermal bonded nonwoven fabric or spunlace nonwoven fabric. However, the material of the skin contact layer 42 and the material of the fluid distribution layer 41 are not limited to the above descriptions.

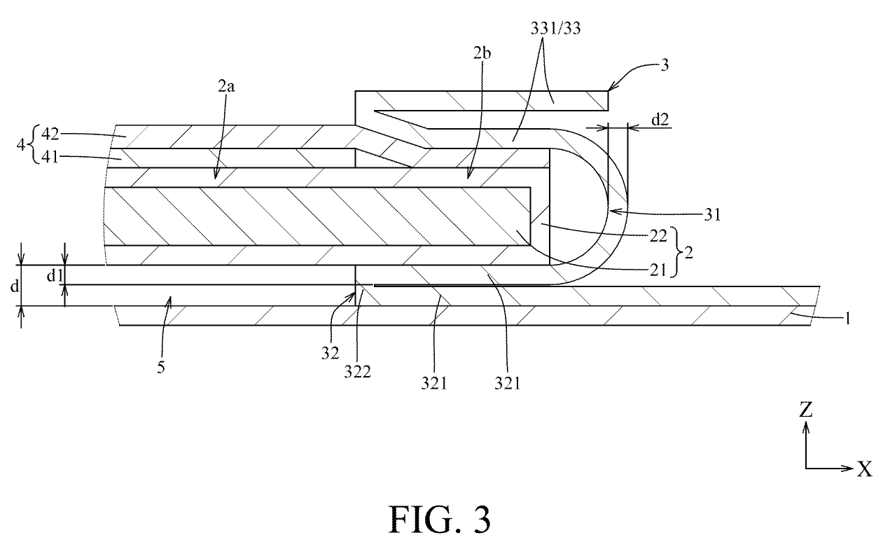

Referring to FIGS. 2 and 3, the folded units 3 are respectively disposed at two opposite sides of the absorbent unit 2 and are used to block the liquid and allow gas to penetrate therethrough. Each of the folded units 3 includes a side barrier structure 31, a supporting structure 32 which is connected to the side barrier structure 31, and a leakproof structure 33 which is connected to the side barrier structure 31 and which is disposed opposite to the supporting structure 32 in the first direction (Z). The side barrier structures 31 of the folded units 3 are respectively located on outer sides of the side portions (2b) which are distal from each other and are adapted to prevent the liquid from flowing outwardly from the absorbent unit 2 in the second direction (X). In the embodiment, the side barrier structure 31 of each of the folded units 3 is in the form of a membrane, and a cross-section of the side barrier structure 31 cut along the second direction (X) is in an arc shape.

The leakproof structure 33 of each of the folded units 3 covers an adjacent one of the side portions (2b). Specifically, the leakproof structures 33 of the folded units 3 are separated from each other in the second direction (X), respectively cover two opposite sides of the skin contact layer 42 which are located on the side portions (2b), and are respectively located on two opposite outer sides of the fluid distribution layer 41. The leakproof structures 33 of the folded units 3 are designed to block the liquid from flowing outwardly in the second direction (X) along the top sheet structure 4 or a surface of the absorbent unit 2 before being absorbed by the absorbent unit 2 so as to prevent liquid leakage. In some embodiments, the leakproof structure 33 of each of the folded units 3 includes two leakproof layers 331 which are arranged in the first direction (Z) and each of which is in the form of a membrane. The leakproof layers 331 of the leakproof structure 33 of each of the folded units 3 are integrally formed in one piece, and are connected to each other at a side which is away from a respective one of the side barrier structures 31 of the folded units 3 in the second direction (X). As such, the leakproof structure 33 of each of the folded units 3 features a folded and double-layered structure, and creates a floating and elevated configuration, thereby further preventing liquid leakage.

The supporting structure 32 of each of the folded units 3 is disposed between the liquid-impermeable underlayer 1 and an adjacent one of the side portions (2b) so that the main portion (2a) is spaced apart from the liquid-impermeable underlayer 1 by a gap 5. The gap 5 is designed to allow the gas (e.g., vapor) to flow therethrough such that vapor of the liquid which is released from the absorbent unit 2 and vapor from the user’s skin may flow outwardly through the gap 5 in the third direction (Y), which improves breathability of the absorbent article, thereby enhancing the user’s comfort during use. Furthermore, by means of the supporting structures 32 of the folded units 3, the main portion (2a) is suspended relative to the liquid-impermeable underlayer 1, which allows the liquid-impermeable underlayer 1 to move relative to the main portion (2a). Thus, the main portion (2a) remains in a same position when the liquid-impermeable underlayer 1 moves with the user’s body motion, thereby improving leakproofness.

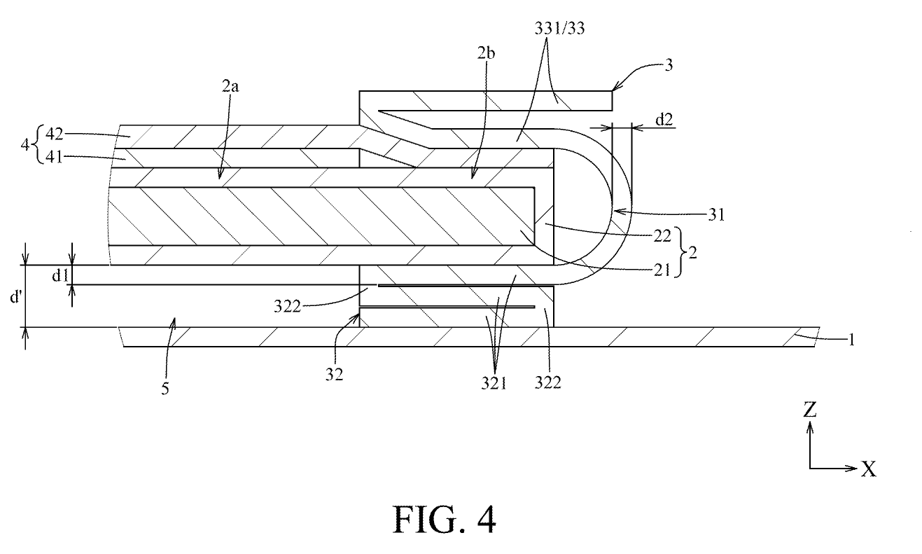

In some embodiments, the supporting structure 32 of each of the folded units 3 includes supporting layers 321 and connecting portions 322. The supporting layers 321 are stacked on one another. The supporting layers 321 and the connecting portions 322 of the supporting structure 32 of each of the folded units 3 are integrally formed in one piece and may be formed in a serpentine shape (see FIG. 4). The connecting portions 322 occupy corners of the serpentine shape and are arranged in an alternating manner in the first direction (Z) (i.e., one connecting portion 322 occupies a right corner in the second direction (X), a next connecting portion 322 occupies a left corner in the second direction (X) that is higher than the right corner in the first direction (Z), and so on and so forth). In certain embodiments, as shown in FIG. 3, the supporting structure 32 of each of the folded units 3 includes two supporting layers 321 so only one connecting portion 322 of the supporting structure 32 of each of the folded units 3 is formed. As such, a structural configuration of the supporting structure 32 of each of the folded units 3 may be considered as a folded and multi-layered structure, and has a function of supporting the absorbent unit 2. Moreover, a distance between the main portion (2a) and the liquid-impermeable underlayer 1, which is a dimension of the gap 5 that is defined by the main portion (2a) and the liquid-impermeable underlayer 1 in the first direction (Z), may be controlled by varying the quantity of the supporting layers 321.

Referring to FIG. 3, in each of the folded units 3, the leakproof structure 33, the side barrier structure 31 and the supporting structure 32 are integrally formed in one piece. Each of the folded units 3 may be a membrane which is folded to form the leakproof structure 33, the side barrier structure 31 and the supporting structure 32. As such, a step of adhering the leakproof structure 33 to the side barrier structure 31 or adhering the side barrier structure 31 to the supporting structure 32 may be eliminated during manufacturing processes, and liquid leakage may be prevented from occurring at joints of each of the folded units 3, thereby improving reliability. Furthermore, in each of the folded units 3, each of the supporting layers 321 and the side barrier structure 31 are both segments of the membrane so that a dimension (i.e., thickness (d1)) of each of the supporting layers 321 in the first direction (Z) is identical to a dimension (i.e., thickness (d2)) of the side barrier structure 31 of each of the folded units 3 in the second direction (X). On the other hand, a material of each of the folded units 3 is a water-repellent material and includes spunbond nonwoven fabric, thermal bonded nonwoven fabric or cotton fabric, but is not limited thereto.

Referring to FIGS. 3 and 4, in some embodiments, a dimension of the supporting structure 32 of each of the folded units 3 in the first direction (Z) (i.e., total thickness) is one to three times the dimension of the side barrier structure 31 of each of the folded units 3 in the second direction (X) (i.e., thickness (d2)). The distance between the main portion (2a) and the liquid-impermeable underlayer 1 is approximately identical to the dimension of the supporting structure 32 of each of the folded units 3 in the first direction (Z) (i.e., total thickness). In the embodiment shown in FIG. 3, in each of the folded units 3, the quantity of the supporting layers 321 is exemplified by two, so a quantity of the connecting portions 322 in the supporting structure 32 is one. In each of the folded units 3, the connecting portion 322 is located on a side of the supporting layers 321 which is distal from the side barrier structure 31 in the second direction (X). In this case, the dimension of the supporting structure 32 in the first direction (Z) (which is approximately identical to the distance (d) between the main portion (2a) and the liquid-impermeable underlayer 1) is two times the dimension of the side barrier structure 31 in the second direction (X) (i.e., thickness (d2), which is approximately identical to the thickness (d1) of each of the supporting layers 321). In the embodiment shown in FIG. 4, in each of the folded units 3, the quantity of the supporting layers 321 is exemplified by three, so the quantity of the connecting portions 322 in the supporting structure 32 is two. In each of the folded units 3, one of the connecting portions 322 which is proximal to the side barrier structure 31 in the first direction (Z) and which connects two corresponding ones of the supporting layers 321 that are adjacent to each other in the first direction (Z) is located on a side of the two corresponding ones of the supporting layers 321 which is distal from the side barrier structure 31 in the second direction (X); another one of the connecting portions 322 which is distal from the side barrier structure 31 in the first direction (Z) and which connects two corresponding ones of the supporting layers 321 that are adjacent to each other in the first direction (Z) is located on a side of the two corresponding ones of the supporting layers 321 which is proximal to the side barrier structure 31 in the second direction (X). In this case, the dimension of the supporting structure 32 in the first direction (Z) (which is approximately identical to the distance (d’) between the main portion (2a) and the liquid-impermeable underlayer 1) is three times the dimension of the side barrier structure 31 in the second direction (X) (i.e., thickness (d2), which is approximately identical to the thickness (d1) of each of the supporting layers 321). Through altering the quantity of the supporting layers 321 of the supporting structure 32 of each of the folded units 3, the dimension of the supporting structure 32 in the first direction (Z) is one to three times the dimension of the side barrier structure 31 in the second direction (X), so that the gap 5 is less likely to be deformed and breathability of the absorbent article may be maintained.

Referring to FIGS. 2 and 3, a proportion of a dimension (L1) of a portion of the supporting structure 32 of each of the folded units 3 overlapping with the absorbent unit 2 in the second direction (X) to a dimension of the absorbent unit 2 in the second direction (X) ranges from 10% to 20%. In some embodiments, a width of one of the supporting layers 321 of the supporting structure 32 of each of the folded units 3 which is bound to a respective one of the side portions (2b) in the second direction (X) is 10% to 20% of a width of the absorbent unit 2 in the second direction (X). In addition, a proportion of a dimension (L2) of a portion of the leakproof structure 33 of each of the folded units 3 overlapping with the absorbent unit 2 in the second direction (X) to the dimension (L) of the absorbent unit 2 in the second direction (X) ranges from 8% to 15%. In some embodiments, a width of one of the leakproof layers 331 of the leakproof structure 33 of each of the folded units 3 which is bound to a respective one of the side portions (2b) in the second direction (X) is 8% to 15% of the width of the absorbent unit 2 in the second direction (X).

When in use, the absorbent article is firstly applied to a user’s body with the top sheet structure 4 facing the user’s skin. Then, most of the liquid from the user is guided to the absorbent unit 2 by the top sheet structure 4, while a small amount of the liquid is blocked by the leakproof structures 33 and the side barrier structures 31 of the folded units 3 so as to prevent liquid leakage. The gap 5, which is defined by the main portion (2a) and the liquid-impermeable underlayer 1, allows the vapor to circulate with the outside, thereby providing breathability.

In summary, by means of the supporting structures 32, the main portion (2a) is spaced apart from the liquid-impermeable underlayer 1 so as to form the gap 5 between the main portion (2a) and the liquid-impermeable underlayer 1 for circulation of the vapor, so that the breathability of the user’s skin is improved, thereby enhancing the user’s comfort during use. Moreover, by means of the supporting structures 32, the main portion (2a) is suspended relative to the liquid-impermeable underlayer 1 so as to allow the main portion (2a) to remain in place when the liquid-impermeable underlayer 1 moves with the user’s movement, thereby indeed achieving purposes of the disclosure.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. An absorbent article, comprising:
a liquid-impermeable underlayer for preventing penetration of a liquid;
an absorbent unit which is disposed on said liquid-impermeable underlayer in a first direction for absorbing the liquid, and which includes a main portion and two side portions that are respectively connected to two opposite sides of said main portion in a second direction that intersects the first direction; and
two folded units each of which includes a side barrier structure and a supporting structure which is connected to said side barrier structure, said side barrier structures of said folded units being respectively located on outer sides of said side portions which are distal from each other and being adapted to prevent the liquid from flowing outwardly from said absorbent unit, said supporting structure of each of said folded units being disposed between said liquid-impermeable underlayer and an adjacent one of said side portions so that said main portion is spaced apart from said liquid-impermeable underlayer by a gap.

2. The absorbent article as claimed in claim 1, wherein said supporting structure of each of said folded units includes supporting layers which are stacked on one another.

3. The absorbent article as claimed in claim 2, wherein said supporting structure of each of said folded units further includes connecting portions, said supporting layers and said connecting portions of said supporting structure of each of said folded units being integrally formed in one piece and being formed in a serpentine shape, said connecting portions occupying corners of the serpentine shape and being arranged in an alternating manner.

4. The absorbent article as claimed in claim 3, wherein each of said folded units further includes a leakproof structure which is connected to said side barrier structure of each of said folded units, said leakproof structure of each of said folded units covering an adjacent one of said side portions, in each of said folded units, said leakproof structure, said side barrier structure and said supporting structure being integrally formed in one piece.

5. The absorbent article as claimed in claim 4, wherein said leakproof structure of each of said folded units includes two leakproof layers which are arranged in the first direction, said leakproof layers being connected to each other at a side which is away from a respective one of said side barrier structures of said folded units in the second direction.

6. The absorbent article as claimed in claim 1, further comprising a top sheet structure which covers said absorbent unit and which is liquid-permeable for guiding the liquid towards said absorbent unit.

7. The absorbent article as claimed in claim 6, wherein each of said folded units further includes a leakproof structure which is connected to said side barrier structure of each of said folded units, said leakproof structure of each of said folded units covering an adjacent one of said side portions, said top sheet structure including a fluid distribution layer which is located between said leakproof structures of said folded units and which is disposed on said absorbent unit, and a skin contact layer which covers said fluid distribution layer and which is connected between said side portions and said leakproof structures of said folded units, said skin contact layer being liquid-permeable and allowing the liquid to penetrate therethrough to said fluid distribution layer, said fluid distribution layer being used to guide the liquid from said skin contact layer towards said absorbent unit.

8. The absorbent article as claimed in claim 7, wherein a material of said skin contact layer includes spunbond nonwoven fabric, thermal bonded nonwoven fabric, a perforated film or cotton fabric, and a material of said fluid distribution layer includes thermal bonded nonwoven fabric or spunlace nonwoven fabric.

9. The absorbent article as claimed in claim 4, wherein said folded units are used to block the liquid and allow gas to penetrate therethrough, a material of each of said folded units including spunbond nonwoven fabric, thermal bonded nonwoven fabric or cotton fabric.

10. The absorbent article as claimed in claim 1, wherein said absorbent unit includes an absorbent body and a wrapping layer which wraps said absorbent body, said absorbent body and said wrapping layer cooperatively defining said main portion and said side portions, a material of said absorbent body including a mixture of superabsorbent polymer and pulp, a material of said wrapping layer including nonwoven fabric or plant fiber.

11. The absorbent article as claimed in claim 6, wherein said top sheet structure is formed with recessed portions which are recessed from an upper surface of said top sheet structure to said absorbent unit.

12. The absorbent article as claimed in claim 1, wherein said liquid-impermeable underlayer includes gas-permeable holes.

13. The absorbent article as claimed in claim 1, wherein a proportion of a dimension of a portion of said supporting structure of each of said folded units overlapping with said absorbent unit in the second direction to a dimension of said absorbent unit in the second direction ranges from 10% to 20%.

14. The absorbent article as claimed in claim 7, wherein a proportion of a dimension of a portion of said leakproof structure of each of said folded units overlapping with said absorbent unit in the second direction to a dimension of said absorbent unit in the second direction ranges from 8% to 15%.

15. The absorbent article as claimed in claim 4, wherein a proportion of a dimension of a portion of said leakproof structure of each of said folded units overlapping with said absorbent unit in the second direction to a dimension of said absorbent unit in the second direction ranges from 8% to 15%.

16. The absorbent article as claimed in claim 1, wherein, in each of folded units, said supporting structure and said side barrier structure are integrally formed in one piece, a dimension of said supporting structure of each of said folded units in the first direction being 1 to 3 times a dimension of said side barrier structure of each of said folded units in the second direction, a distance between said main portion and said liquid-impermeable underlayer being approximately identical to the dimension of said supporting structure of each of said folded units in the first direction.
